(19) United States
(12) Patent Application Publication
(10) Pub. No.: US 2026/0014189 A1
(43) Pub. Date: Jan. 15, 2026

(54) PHARMACEUTICAL COMPOSITION FOR PREVENTING OR TREATING NEOPLASTIC DISORDERS COMPRISING AURANOFIN AS AN ACTIVE INGREDIENT

(71) Applicant: SOONCHUNHYANG UNIVERSITY INDUSTRY ACADEMY COOPERATION FOUNDATION, Chungcheongnam-do (KR)

(72) Inventors: Jong Dae Lee, Seoul (KR); Sin-Aye Park, Chungcheongnam-do (KR); Wan Namkung, Incheon (KR)

(21) Appl. No.: 19/032,593

(22) Filed: Jan. 21, 2025

(30) Foreign Application Priority Data
Jul. 11, 2024 (KR) 10-2024-0091599

(51) Int. Cl. A61K 31/7135; A61P 35/00

(52) U.S. Cl. CPC A61K 31/7135; A61P 35/00

(57) ABSTRACT

The present disclosure relates to a pharmaceutical composition comprising auranofin as an active ingredient for preventing or treating neoplastic disorders and, more specifically, to a pharmaceutical composition comprising auranofin as an active ingredient for preventing or treating neoplastic disorders and a method for preventing or treating neoplastic disorders using a composition containing auranofin.

### CROSS-REFERENCE TO RELATED APPLICATION

This patent application claims the benefit of and priority to Korean Patent Application No. 10-2024-0091599, filed with the Korean Intellectual Property Office on Jul. 11, 2024, the disclosure of which is incorporated herein by reference.

### FIELD

The present disclosure was carried out with the support of the Ministry of Science and ICT, under project identification number 1711197632 and subproject number 00239096. The research management organization for the project is the National Research Foundation of Korea, the project title is “Basic Individual Research (Ministry of Science and ICT)”, and the research project name is “Discovery and Mechanism Study of Vestibular Schwannoma Therapeutics Using Drug Repurposing”. The principal institution is Soonchunhyang University, and the research period is from Jun. 1, 2023, to Feb. 29, 2024.

The present disclosure relates to a pharmaceutical composition including auranofin as an active ingredient for preventing or treating neoplastic disorders and, more particularly, to a pharmaceutical composition including auranofin as an active ingredient for preventing or treating neoplastic disorders and a method for preventing or treating neoplastic disorders using a composition including auranofin.

### BACKGROUND

The human body has 12 pairs of nerves originating from the brain and 31 pairs of nerves originating from the spinal cord, which extend to various parts of the body through pathways in the skull and vertebrae. Schwannomas are tumors that arise from the nerve sheath, a tubular structure supporting these nerves, specifically from Schwann cells. Schwannomas typically occur in narrow regions where nerves originating from the brain or spinal cord pass through bony pathways. Occasionally, schwannomas may also arise in peripheral nerves or soft tissues. When schwannomas occur in cranial nerves, they appear as brain tumors, and about 70-80% of these cases develop in the vestibulocochlear nerve, thus being commonly referred to as vestibular schwannomas. When occurring in the spinal cord, schwannomas are referred to as spinal schwannomas. In brain tumors, schwannomas are often benign, although rarely malignant, allowing for surgical removal as the primary treatment. Schwannomas have a slow proliferation rate and rarely metastasize to organs outside the brain. However, they may recur even after complete resection. If the tumor grows large or invades surrounding brain tissue, cranial nerves, or blood vessels, surgical removal becomes difficult, and the risk of complications increases. Schwannomas, including malignant brain tumors, account for approximately 8% of all brain tumors, are more prevalent in women, and primarily occur in individuals aged 30 to 60 years.

Neurofibromatosis (NF), which is a genetic disorder affecting bones, soft tissues, skin, and the nervous system, is classified into neurofibromatosis type 1 (NF1) and neurofibromatosis type 2 (NF2). Neurofibromatosis type 2 is characterized by benign tumors developing in the eighth cranial nerve, resulting in symptoms such as hearing loss, tinnitus, and balance impairment. Because these tumors occur within Schwann cells in the central nervous system, they are also referred to as vestibular schwannomas. Neurofibromatosis type 2 typically manifests between the ages of 18 and 24, and by age 30, almost all patients develop bilateral vestibular schwannomas. In addition, tumors such as meningiomas, ependymomas, and, in rare cases, astrocytomas can progress in the cranial and peripheral nerves.

Drug repositioning is a strategy that identifies new therapeutic uses for existing drugs that are already approved for safety or have failed clinical trials due to efficacy issues. This approach significantly shortens the drug development timeline from over 10 years while also reducing the risks associated with drug safety.

Through drug repositioning for vestibular schwannoma, which is a type of schwannoma, gene expression data confirmed for vestibular schwannoma was applied to FDA-approved drugs on the basis of computational algorithms, reporting the research result that mifepristone is effective against vestibular schwannoma cell line HEI-193. In addition, studies have shown that aspirin may inhibit the growth of vestibular schwannomas, with clinical trials involving 300 patients ongoing for evaluating effects of aspirin on vestibular schwannoma since 2018. However, both drugs require high doses to achieve therapeutic effects, leading to concerns about side effects and toxicity, compromising their safety and efficacy.

Auranofin (C₂₀H₃₄AuO₉PS) is a gold phosphine compound used to treat rheumatoid arthritis. Recent studies have revealed its efficacy in treating malignant brain tumors, such as glioblastoma. However, the effects of auranofin on schwannomas or neurofibromatosis have not yet been demonstrated.

The current primary treatment methods for schwannomas include surgery, radiation therapy, and observation. Complete surgical resection is the most effective option, but it is limited by factors such as the patient's age, general health, symptom severity, and the size and location of the tumor. Furthermore, surgical procedures carry risks and potential complications. Although schwannomas can cause severe conditions such as hearing loss, sensory deficits, ataxia, balance dysfunction, and brainstem compression leading to death, they are classified as benign tumors rather than cancer, preventing approval for chemotherapeutic treatments. Therefore, there is an urgent need to develop new treatments that provide high therapeutic efficacy at low doses without adverse effects.

### SUMMARY

To address the aforementioned issues, the present inventors identified a drug with therapeutic efficacy against neoplastic disorders through drug repositioning and completed the development of a pharmaceutical composition including auranofin as an active ingredient for preventing or treating neoplastic disorders, which leads to the present disclosure.

Accordingly, an aspect of the present disclosure is to provide a pharmaceutical composition including auranofin as an active ingredient for preventing or treating neoplastic disorders.

Another aspect of the present disclosure is to provide a method for preventing or treating neoplastic disorders, the method including administering a composition containing auranofin to a subject other than humans.

The present disclosure relates to a pharmaceutical composition including auranofin as an active ingredient for preventing or treating neoplastic disorders. More specifically, the present disclosure relates to a pharmaceutical composition including auranofin as an active ingredient for the prevention or treatment of neoplastic disorders and a method for preventing or treating neoplastic disorders using a composition containing auranofin.

Below, a detailed description will be given of the present disclosure.

An aspect of the present disclosure pertains to a pharmaceutical composition for preventing or treating neoplastic disorders, comprising auranofin as an active ingredient.

The neoplastic disorder may include at least one selected from the group consisting of schwannoma and neurofibromatosis type 2 syndrome, but is not limited thereto.

The schwannoma may include vestibular schwannoma, but this is not limited thereto.

The composition may contain auranofin at a concentration of 0.1 to 5 mg/mL, and preferably at a concentration of 0.1 to 3 mg/ml, 0.1 to 1 mg/ml, 0.2 to 5 mg/mL, or 0.2 to 3 mg/mL, for example, 0.2 to 1 mg/mL, but with no limitations thereto.

Auranofin may be administered at a dosage of 1 to 20 mg per kg of body weight, and preferably at a dosage of 1 to 15 mg, 1 to 10 mg, 2 to 20 mg, or 2 to 15 mg per kg of body weight. For example, auranofin may be administered at a dosage of 2 to 10 mg per kg of body weight, but is not limited thereto.

The composition may be administered 2 to 5 times per week to a subject, preferably 2 to 3 times per week or 3 to 5 times per week. For example, the composition may be administered 3 times per week.

If the administration frequency is less than twice per week, the prophylactic and therapeutic effect may be minimal. If the administration exceeds five times per week, toxicity may occur in the subject.

The pharmaceutical composition of the present disclosure may include a pharmaceutically effective amount of a combination powder and/or a pharmaceutically acceptable carrier.

The term “pharmaceutically effective amount”, as used herein, refers to an amount sufficient to achieve the aforementioned efficacy or activity of the combination powder.

The pharmaceutically acceptable carrier included in the pharmaceutical composition of the present disclosure may be conventionally used in formulation preparation and may include, but is not limited to, lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oil. The pharmaceutical composition of the present disclosure may further include lubricants, humectants, sweeteners, flavoring agents, emulsifiers, suspending agents, and preservatives in addition to the above components.

The pharmaceutical composition according to the present disclosure may be administered to mammals, including humans, via various routes. The administration route may be any conventional method, including oral, transdermal, intravenous, intramuscular, or subcutaneous administration, and preferably via oral administration.

The appropriate dosage of the pharmaceutical composition of the present disclosure may vary depending on factors such as formulation method, administration mode, patient age, weight, gender, pathological condition, diet, time of administration, administration route, excretion rate, and responsiveness. A skilled physician can readily determine and prescribe the effective dose required for the desired prevention or treatment.

The pharmaceutical composition of the present disclosure may be formulated, together with a pharmaceutically acceptable carrier and/or excipient, according to a method that could be easily practiced by a person skilled in the art to which the present invention pertains, and the composition of the present disclosure may be prepared into a unit dosage form or may be contained in a multi-dose container. In this regard, the formulation may take the form of a solution, suspension, or emulsion in an oily or aqueous medium, an extract, a pulvis, granules, a tablet, or a capsule, or a gel (e.g., hydrogel), and may further comprise a dispersant or a stabilizer.

Another aspect of the present disclosure relates to a method for preventing or treating neoplastic disorders, the method including administering a composition containing auranofin to a subject.

The subject may be a mammal, including non-human mammals, but is not limited thereto.

The neoplastic disorder may include at least one selected from the group consisting of schwannoma and neurofibromatosis type 2 syndrome, but is not limited thereto.

The schwannoma may include vestibular schwannoma, but this is not limited thereto.

The composition may contain auranofin at a concentration of 0.1 to 5 mg/mL, preferably at a concentration of 0.1 to 3 mg/mL, 0.1 to 1 mg/mL, 0.2 to 5 mg/mL, or 0.2 to 3 mg/mL, for example, 0.2 to 1 mg/mL, but with no limitations thereto.

The auranofin may be administered at a dosage of 1 to 20 mg per kg of body weight, preferably 1 to 15 mg, 1 to 10 mg, 2 to 20 mg, or 2 to 15 mg per kg of body weight. For example, it may be administered at 2 to 10 mg per kg of body weight, but is not limited thereto.

The composition may be administered 2 to 5 times per week to a subject, preferably 2 to 3 times per week or 3 to 5 times per week. For example, it may be administered 3 times per week.

The present disclosure relates to a pharmaceutical composition including auranofin as an active ingredient for preventing or treating neoplastic disorders. Exhibiting excellent tumor growth inhibition effects against neoplastic disorders such as schwannoma and neurofibromatosis type 2 syndrome, the composition can find applications as a pharmaceutical composition for preventing or treating neoplastic disorders.

### BRIEF DESCRIPTION OF THE DRAWINGS

The above and other aspects, features and advantages of the present disclosure will be more apparent from the following detailed description taken in conjunction with the accompanying drawings, in which:

FIG. 1 shows plots illustrating the cytotoxicity results of an FDA-approved drug library screened through high-throughput screening (HTS) for schwannoma cell lines and neurofibromatosis type 2 cell lines according to an embodiment of the present disclosure;

FIG. 2 shows graphs showing the IC₅₀ values of the drugs identified through drug repositioning for schwannoma cell lines and neurofibromatosis type 2 cell lines according to an embodiment of the present disclosure;

FIG. 3 shows graphs comparing the cytotoxicity and IC₅₀ values of auranofin, aspirin, and mifepristone for schwannoma cell lines and neurofibromatosis type 2 cell lines according to an embodiment of the present disclosure;

FIG. 4 shows photographic images illustrating the changes in the expression of apoptosis markers after treating schwannoma cell lines and neurofibromatosis type 2 cell lines with auranofin, according to an embodiment of the present disclosure;

FIG. 5a is a photographic image of mice with induced tumor formation divided into groups based on auranofin dosage, according to an embodiment of the present disclosure;

FIG. 5b is a photographic image comparing the tumor sizes in mice with induced tumor formation after administering auranofin, according to an embodiment of the present disclosure; and

FIG. 5c is a plot comparing the tumor sizes in mice with induced tumor formation after administering auranofin, according to an embodiment of the present disclosure.

### DETAILED DESCRIPTION

Hereinafter, a better understanding of the present disclosure may be obtained through the following examples, which are set forth to illustrate, but are not to be construed to limit, the present disclosure.

### Example 1: Culture of Schwannoma and Neurofibromatosis

### Type 2 Cell Lines

Schwannoma cell line RT4-D6P2T (RT4) and neurofibromatosis type 2 cell line SC4 were cultured in 60-mm² culture dishes until approximately 50% confluency. Cells were serum-starved for 6 hours in FBS-free DMEM medium.

The RT4 cell line was purchased from the American Type Culture Collection (CRL-2768), and the SC4 cell line was obtained from the House Ear Institute (Los Angeles, CA, USA). Both RT4 and SC4 cells were cultured in DMEM medium containing 10% FBS and 100 units/ml of penicillin/streptomycin at 37° C. with 5% CO₂.

### Example 2: Identification of Novel Drugs Through Drug Repositioning

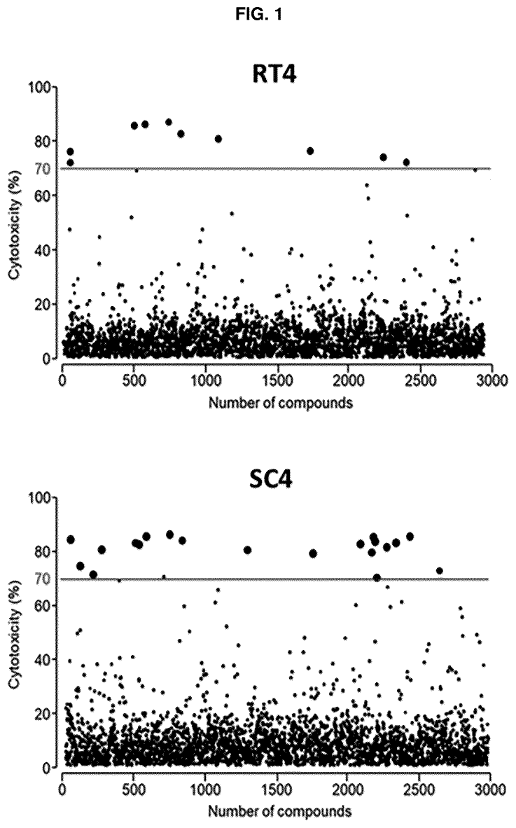

To discover effective therapeutics for schwannoma and neurofibromatosis type 2 using drug repositioning, a library of 2,920 FDA-approved drugs was screened for cytotoxicity against the RT4 and SC4 cell lines using high-throughput screening (HTS). As shown in FIG. 1, the 2,920 drugs were applied at a concentration of 10 UM to RT4 and SC4 cell lines for 24 hours. Selection was made of drugs exhibiting cytotoxicity exceeding 70% in both cell lines. As a result, a total of eight drugs including auranofin was identified.

### Example 3: Measurement of Cell Viability for Schwannoma Cell Line

The IC₅₀ values of the eight drugs identified through Example 2 were measured for the RT4 schwannoma cell line. The RT4 cells cultured according to Example 1 were treated with each drug at varying concentrations for 24 hours, and the concentration resulting in 50% cell viability was measured. The results are summarized in Table 1.

TABLE 1

| Name of Compound | IC₅₀ (μM) |
|---|---|
| Auranofin (C1) | 0.23 |
| C2 | 4.05 |
| C3 | 7.83 |
| C4 | 2.99 |
| C5 | 2.20 |
| C6 | 0.41 |
| C7 | 2.77 |
| C8 | 4.99 |

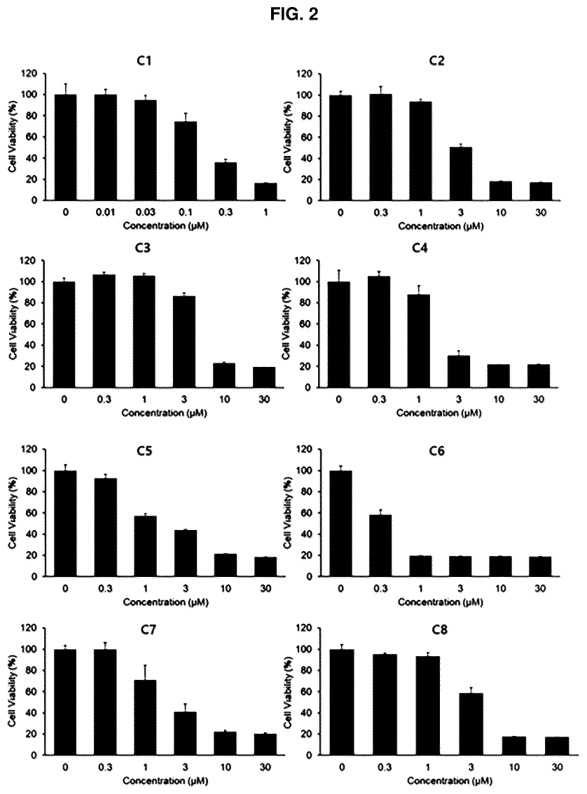

As shown in FIG. 2, all eight drugs exhibited low IC₅₀ values for the RT4 cell line, with auranofin demonstrating the lowest IC₅₀ value of 0.23 UM. The data indicates that all the eight drugs can inhibit the survival of the schwannoma cell line at low concentrations.

### Example 4: Comparison with Existing Drugs in Terms of Cell Viability

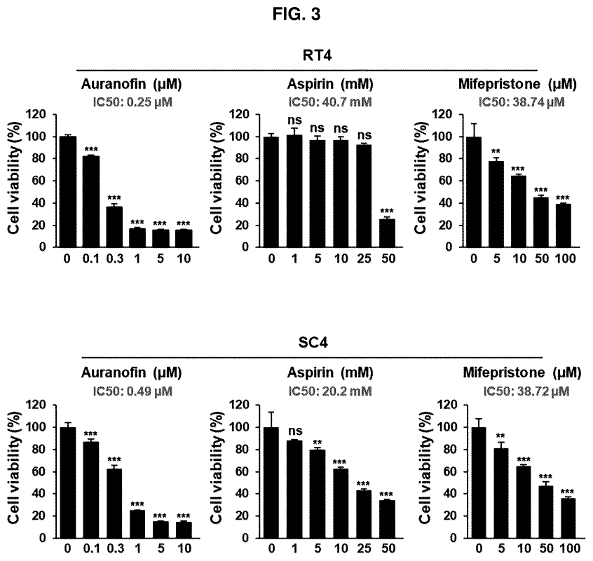

Among the eight drugs, auranofin, which had the lowest IC₅₀ value for RT4 cells, was further evaluated for efficacy. RT4 and SC4 cell lines were treated with auranofin, aspirin, and mifepristone at varying concentrations for 24 hours. The results are shown in Table 2 and FIG. 3.

TABLE 2

| | IC₅₀ | | |
|---|---|---|---|
| Name of Compound | | RT4 | SC4 |

| | | | | |
|---|---|---|---|---|
| Auranofin (C1) | 0.25 | μM | 0.49 | μM |
| Aspirin | 40.7 | mM | 20.2 | mM |
| Mifepristone | 38.74 | μM | 38.72 | μM |

Auranofin's IC₅₀ values for RT4 and SC4 cells were 0.25 UM and 0.49 μM, respectively, significantly lower than those of aspirin and mifepristone.

### Example 5: Measurement of Change in Apoptosis Marker Expression by Auranofin Treatment

To measure changes in apoptosis marker expression induced by auranofin treatment, RT4 and SC4 cells cultured according to Example 1 were treated with auranofin at concentrations of 0, 0.1, and 0.3 μM for 24 hours. The protein expression of apoptosis markers cleaved PARP and cleaved caspase-3 was compared using Western blotting.

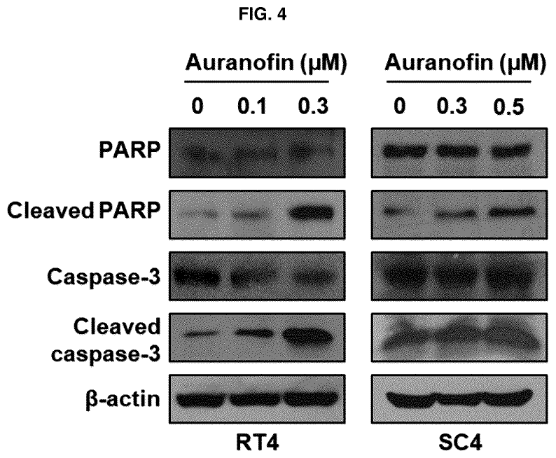

As shown in FIG. 4, treatment of RT4 and SC4 cell lines with auranofin led to a dose-dependent increase in the expression of cleaved PARP and cleaved caspase-3 in both RT4 and SC4 cell lines.

### Example 6: Tumor Growth Inhibition Effect of Auranofin in Tumor-Induced Mice

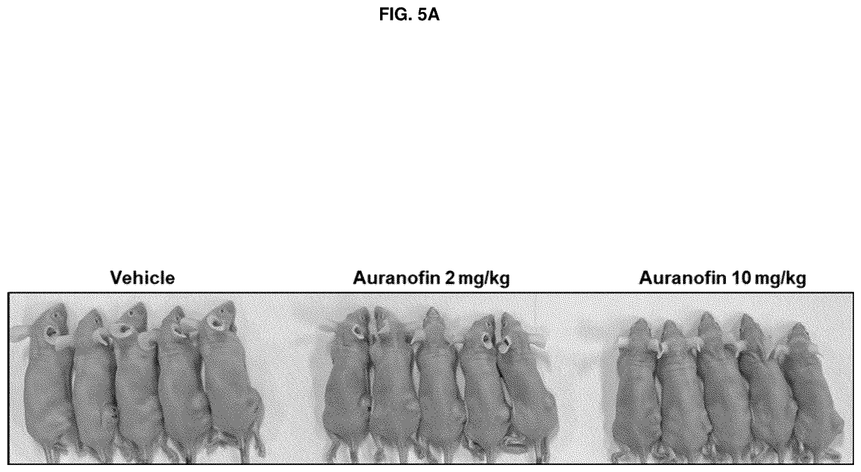

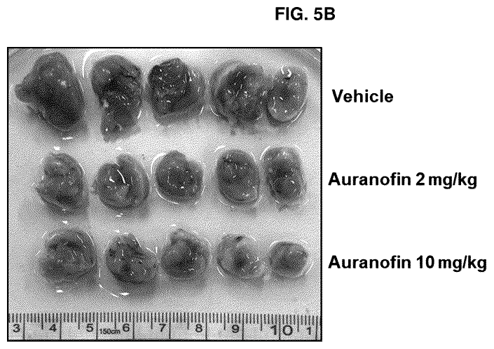

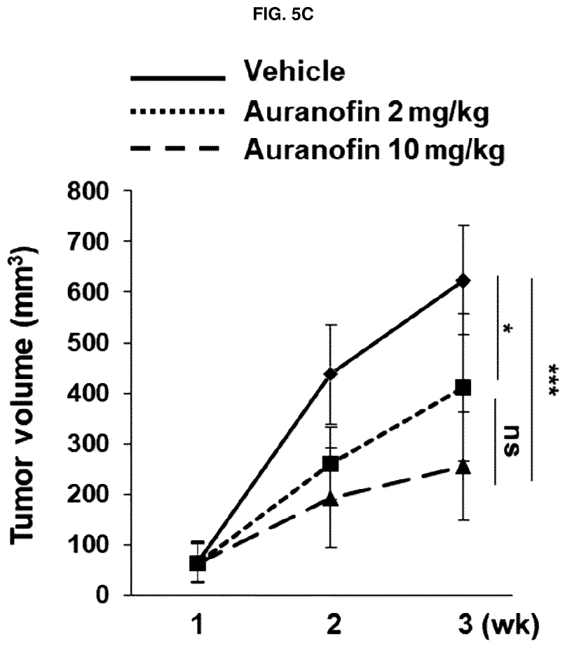

As shown in FIG. 5a, 6-week-old female Balb/c nude mice with an average body weight of approximately 18 g were divided into three groups based on auranofin dosage. SC4 cells were injected into the flank of the mice to induce tumor formation.

For the low-dose group (2 mg/kg), 0.04 mg of powdered auranofin was dissolved in a solvent mixture of DMSO (20 μL, 10%), PEG400 (40 μL, 20%), and PBS (140 μL) to make a total volume of 200 μL per injection. For the high-dose group (10 mg/kg), 0.2 mg of powdered auranofin was dissolved in the same solvent mixture of DMSO 20 μL (10%), PEG400 40 μL (20%), and PBS 140 μL to make a total volume of 200 μL which was then injected per 20 g of mice. Auranofin was intraperitoneally administered at a dose of 2 mg/kg or 10 mg/kg three times per week for two weeks to mice, and the control group received only the solvent mixture of DMSO (10%), PEG400 (20%), and PBS. Tumor sizes were compared, and the results are summarized in Table 3.

TABLE 3

| | Week 1 | Week 2 | Week 3 |
|---|---|---|---|
| Control | 66.34 mm³ | 437.78 mm³ | 623.78 mm³ |
| Auranofin (C1) 2 mg/kg | 64.60 mm³ | 261.49 mm³ | 410.80 mm³ |
| Auranofin (C1) 10 mg/kg | 64.01 mm³ | 193.38 mm³ | 256.02 mm³ |

As shown in FIGS. 5b and 5c, and Table 3, tumor growth in the auranofin-treated groups was significantly inhibited compared to the control group. Specifically, the 10 mg/kg auranofin-treated group showed a tumor growth rate approximately 42.5% lower than the control group, indicating effective tumor growth suppression.

What is claimed is:

1. A method for preventing or treating a neoplastic disorder in a subject in need thereof, the method comprising:
administering to the subject a composition containing auranofin.

2. The method of claim 1, wherein the neoplastic disorder is at least one selected from the group consisting of schwannoma and neurofibromatosis type 2 syndrome.

3. The method of claim 2, wherein the schwannoma is vestibular schwannoma.

4. The method of claim 1, wherein the composition contains auranofin at a concentration of 0.1 to 5 mg/mL.

5. The method of claim 1, wherein the auranofin is administered at a dose of 1 to 20 mg per kg of body weight of a subject.

6. The method of claim 1, wherein the composition containing auranofin is administered 2 to 5 times per week.
